(19) United States
(12) Patent Application Publication
(10) Pub. No.: US 2011/0313193 A1
(43) Pub. Date: Dec. 22, 2011

(54) Purification of acetic acid

(76) Inventors: Noel C. Hallinan, Loveland, OH (US); Brian A. Salisbury, Oxford, PA (US); Wayne J. Brtko, Glen Mills, PA (US)

(21) Appl. No.: 12/802,972

(22) Filed: Jun. 17, 2010

(51) Int. Cl. C07C 51/12; C07C 51/42

(52) U.S. Cl. 562/519; 562/512

(57) ABSTRACT

The invention is a method of purifying acetic acid containing 15 to 100 ppb iodide compounds. The method comprises contacting the acetic acid in the liquid phase with a phosphine-functionalized support, and recovering a purified acetic acid product having 10 ppb, or less, iodide compounds.

### FIELD OF THE INVENTION

This invention relates to the removal of organic iodides from acetic acid.

### BACKGROUND OF THE INVENTION

Acetic acid is a well-known chemical that is available from Lyondell Chemical Company and other producers. Acetic acid is commercially produced by methanol carbonylation in the presence of a rhodium catalyst, methyl iodide, methyl acetate, and water (the “Monsanto process”), see U.S. Pat. No. 3,769,329. Catalyst stabilizers such as lithium iodide or pentavalent Group VA oxides may also be added to the carbonylation reaction. The process results in a high selectivity to acetic acid. Unfortunately, the presence of iodide in the carbonylation process results in iodide impurities in the acetic acid product. The iodide impurities typically comprise methyl iodide, hexyl iodide, and iodobenzene. For most applications, it is necessary to reduce the amount of iodide impurities to less than 10 ppb. Although distillation can increase acetic acid purity level to 99.5% or greater, it is difficult to reduce iodide impurities to less than 10 ppb by distillation alone without incurring unreasonably high costs.

Many methods have been developed to remove these iodide impurities from acetic acid. Previous disclosed methods include the liquid-phase extraction of the iodide impurities. U.S. Pat. No. 4,908,477, for example, discloses a process wherein the iodide impurities are removed from an acetic acid process stream by liquid phase extraction with a non-aromatic hydrocarbon.

A widely disclosed method teaches removal of iodide impurities by adsorption. A variety of solid adsorbents have been described, and typically contain reactive metals, such as silver, mercury, copper, lead, thallium, palladium, to remove iodide from solution. U.S. Pat. Nos. 4,615,806 and 5,139,981, and 5,227,524 disclose the use of macroreticulated, strong acid cationic exchange resins that contain silver or mercury. The iodide reacts with the resin bound metal and is removed from the acetic acid stream. U.S. Pat. No. 5,220,058 discloses the use of ion exchange resins having metal exchanged thiol functional groups to remove iodide impurities from acetic acid and/or acetic anhydride. The thiol functionality of the ion exchange resin is taught to have been exchanged with silver, palladium, or mercury. European Patent No. 685,445 teaches contacting an iodide containing acetic acid stream with a polyvinylpyridine at elevated temperatures to remove the iodides. U.S. Pat. No. 3,943,229 discloses removing iodide compounds from gaseous streams by contact with certain cross-linked acrylic anion exchange resins.

One problem associated with the use of metal containing adsorbents is that the metals may leach at higher temperature, and may require the use of an additional unexchanged adsorbent bed downstream to catch any leached metal and prevent contamination of the acetic acid product.

U.S. Pat. No. 4,650,615 teaches the purification of carboxylic acid anhydrides, such as acetic anhydride, contaminated with halogen and halide values by treating the anhydrides with a phenyl or an alkyl phosphine in the absence of copper, zinc, silver, and cadmium or their compounds and distilling to recover purified anhydrides.

In sum, new methods for the purification of acetic acid containing iodine impurities are needed. We have discovered an effective, convenient method to purify acetic acid.

### SUMMARY OF THE INVENTION

The invention is a method for purifying acetic acid containing 15 to 100 ppb iodide compound impurities. The method comprises contacting the acetic acid in the liquid phase with a phosphine-functionalized support, and recovering a purified acetic acid product having 10 ppb, or less, iodide impurities. The acetic acid containing 15 to 100 ppb iodide compounds may be produced by reacting methanol and carbon monoxide to produce a crude acetic acid effluent, and then distilling the crude acetic acid effluent in one or more distillation steps.

### DETAILED DESCRIPTION OF THE INVENTION

Acetic acid is a well-known chemical that is available from Lyondell Chemical Company and other producers. The acetic acid having an iodide compound content of 15-100 ppb, and preferably 15-60 ppb, may be produced by any known process, but is preferably the product of the carbonylation of methanol in the presence of a catalyst and methyl iodide (the “Monsanto process”). The methanol carbonylation reaction to produce acetic acid is described in U.S. Pat. No. 3,769,329.

Methanol and carbon monoxide are fed to a carbonylation reactor. The methanol feed to the carbonylation reaction can come from a syngas-methanol facility or any other source. Suitable catalysts include rhodium and iridium catalysts. Rhodium catalysts are preferred. Suitable rhodium catalysts are taught, for example, by U.S. Pat. No. 5,817,869. Typical rhodium catalysts include rhodium metal and rhodium compounds. Preferably, the rhodium compounds are selected from the group consisting of rhodium salts, rhodium oxides, rhodium acetates, organo-rhodium compounds, coordination compounds of rhodium, and mixtures thereof. More preferably, the rhodium compounds are selected from the group consisting of Rh₂(CO)₄I₂, Rh₂(CO)₄Br₂, Rh₂(CO)₄Cl₂, Rh(CH₃CO₂)₂, Rh(CH₃CO₂)₃, [H]Rh(CO)₂I₂, and mixtures thereof.

Suitable iridium catalysts are taught, for example, by U.S. Pat. No. 5,932,764. Typical iridium catalysts include iridium metal and iridium compounds. Examples of iridium compounds include IrCl₃, IrI₃, IrBr₃, [Ir(CO)₂I]₂, [Ir(CO)₂Cl]₂, [Ir(CO)₂Br]₂, [Ir(CO)₄I₂]⁻H⁺, [Ir(CO)₂Br₂]^{-H}⁺, [Ir(CO)₂I₂]⁻H⁺, [Ir(CH₃)I₃(CO)₂]⁻H⁺, Ir₄(CO)₁₂, IrCl₃·4H₂O, IrBr₃·4H₂O, Ir₃(CO)₁₂, Ir₂O₃, IrO₂, Ir(acac)(CO)₂, Ir(acac)₃, Ir(OAc)₃, [Ir₃O(OAc)₆(H₂O)₃][OAc], and H₂[IrCl₆]. Preferably, the iridium compounds are selected from the group consisting of acetates, oxalates, acetoacetates, the like, and mixtures thereof. More preferably, the iridium compounds are acetates.

The iridium catalyst is preferably used with a co-catalyst. Preferred co-catalysts include metals and metal compounds selected from the group consisting of osmium, rhenium, ruthenium, cadmium, mercury, zinc, gallium, indium, and tungsten, their compounds, the like, and mixtures thereof. More preferred co-catalysts are selected from the group consisting of ruthenium compounds and osmium compounds. Most preferred co-catalysts are ruthenium compounds. Preferably, the co-catalysts are acetates.

The carbonylation reaction is performed in the presence of methyl iodide. Preferably, the concentration of methyl iodide is from about 0.6 wt % to about 36 wt % based on the total weight of the reaction medium. More preferably, the concentration of methyl iodide is from about 4 wt % to about 24 wt %. Most preferably, the concentration of methyl iodide is from about 6 wt % to about 20 wt %. Alternatively, methyl iodide can be generated in the carbonylation reactor by adding hydrogen iodide (HI).

Preferably, the carbonylation reaction is performed in the presence of a catalyst stabilizer. Suitable catalyst stabilizers include metal iodide salts such as lithium iodide or non-salt stabilizers such as pentavalent Group VA oxides. See, for example, U.S. Pat. No. 5,817,869. Phosphine oxides are more preferred. Triphenylphosphine oxides are most preferred.

The carbonylation reaction is preferably performed in the presence of water. Preferably, the concentration of water is from about 2 wt % to about 14 wt % based on the total weight of the reaction medium, more preferably from about 2 wt % to about 10 wt %, and most preferably from about 4 wt % to about 8 wt %.

The carbonylation reaction is preferably performed in the presence of methyl acetate. Methyl acetate can be formed in situ or can be added as a starting material to the reaction mixture. Preferably, the concentration of methyl acetate is from about 2 wt % to about 20 wt % based on the total weight of the reaction medium, more preferably from about 2 wt % to about 16 wt %, and most preferably from about 2 wt % to about 8 wt %. Hydrogen may also be fed into the carbonylation reactor. Addition of hydrogen can enhance the carbonylation efficiency. Preferably, the concentration of hydrogen is from about 0.1 mol. % to about 5 mol. % of carbon monoxide in the reactor, and more preferably from about 0.3 mol. % to about 3 mol. %.

The carbonylation reaction is preferably performed at a temperature within the range of about 150° C. to about 250° C., and more preferably within the range of about 150° C. to about 200° C. The carbonylation reaction is preferably performed under a pressure within the range of about 200 psig to about 2,000 psig, and more preferably within the range of about 300 psig to about 500 psig.

The methanol carbonylation reaction produces a crude acetic acid reaction effluent. The crude acetic acid reaction effluent is subjected to one or more distillation steps in order to produce an acetic acid product stream.

Crude acetic acid, as formed by the carbonylation of methanol in the presence of a catalyst and methyl iodide contains various impurities, including iodide compounds. These iodide compound impurities include methyl iodide, butyl iodide, hexyl iodide, iodobenzene, and mixtures thereof.

Following the carbonylation of methanol, the crude acetic acid product mixture is preferably first flash distilled. The flash distillation step separates acetic acid, methanol, and methyl iodide as a vapor fraction from a liquid fraction comprising the catalyst and the catalyst stabilizer, if utilized. The liquid fraction is preferably recycled back to the methanol carbonylation reaction.

The overhead vapor acetic acid-containing stream is preferably further dried and then distilled in a series of distillations to produce an acetic acid product stream. Distillation can produce purified acetic acid containing low levels of iodide compounds, typically 15-100 ppb (and preferably 15-60 ppb) iodide compounds. However, in order to produce acetic acid having an iodide compound content of 10 ppb or less, the distillation process has very high energy requirements that make distillation alone prohibitively expensive. Thus, acetic acid is first preferably distilled to an iodide compound content of 15-100 ppb and then iodide compounds are lowered to 10 ppb or less through a less costly process.

Thus, preferably the method of the invention comprises first reacting methanol and carbon monoxide to produce a crude acetic acid effluent, and then distilling the crude acetic acid effluent in one or more distillation steps to produce an acetic acid stream containing 15 to 100 ppb iodide compounds, and more preferably 15-60 ppb.

According to the method of the invention, acetic acid containing 15 to 100 ppb iodide compounds is contacted in the liquid phase with a phosphine-functionalized support, and recovered to produce a purified acetic acid product having 10 ppb, or less, by weight, iodide compounds, preferably 5 ppb or less. In accordance with the present invention, the impure acetic acid is contacted in the liquid phase with a phosphine-functionalized support adsorbent whereby iodide compound impurities are retained on the phosphine-functionalized support and a purified acetic acid product reduced in iodide compound impurities content is conveniently separated.

The phosphine-functionalized support useful in the invention is a solid material that consists of a support which has been functionalized with a phosphine group. Supports are well-known in the art. There are no particular restrictions on the types of support that are used. For instance, the support can be inorganic oxides, inorganic chlorides, carbon, and organic polymer resins. Preferred inorganic oxides include oxides of Group 2, 3, 4, 5, 6, 13, or 14 elements. Particularly preferred inorganic oxide supports include silica, alumina, titania, zirconia, ceria, niobium oxides, tantalum oxides, molybdenum oxides, tungsten oxides, amorphous titania-silica, amorphous zirconia-silica, amorphous niobia-silica, ceria-silica, and the like. Preferred organic polymer resins include polystyrene, styrene-divinylbenzene copolymers, crosslinked polyethyleneimines, and polybenzimidazole. Suitable supports also include organic polymer resins grafted onto inorganic oxide supports, such as polyethylenimine-silica. Preferably, the support is an inorganic oxide. Particularly preferred inorganic oxide supports include silica, alumina, titania, zirconia, niobium oxides, tantalum oxides, titania-silica, zirconia-silica, alumina-silica, niobia-silica, and the like. Silica, alumina, titania, zirconia, titania-silica, zirconia-silica, and alumina-silica are most preferred.

The phosphine unit of the phosphine-functionalized support is preferably a tertiary phosphine. Preferred tertiary phosphines include trialkylphosphines, dialkylarylphosphines, alkyldiarylphosphines, and triarylphosphines. Alkyldiarylphosphines and triarylphosphines are particularly preferred. Most preferred include methyldiphenylphosphine, ethyldiphenylphosphine, n-propyldiphenylphosphine, n-butyldiphenylphosphine, and triphenylphosphine. Specific commercially available phosphine-functionalized supports include diphenylphosphinoethyl-functionalized silica gel and diphenylphosphine polystyrene resin (both available from Sigma-Aldrich).

In general, suitable phosphine-functionalized supports are further characterized by having a relatively large surface area in relation to their mass. The phosphine-functionalized supports for purpose of this invention preferably have a surface area of at least 100 m²/g, and more preferably the average surface area is from 400 m²/g to 1500 m²/g.

Phosphine-functionalized supports may be prepared by any suitable method. For instance, a lithiated phosphine such as diphenylphosphine lithium (Ph₂PLi) may be reacted with resin-bound phenyl halide to give a phosphine-functionalized support (triphenylphosphine resin). Phosphine-functionalized supports may also be formed by the copolymerization of styrene, divinylbenzene, and a phosphine-substituted styrene such as diphenylphosphinostyrene. An inorganic oxide-functionalized support can be prepared by reacting an inorganic oxide such as silica with a phosphinoalkylsilane compound such as diphenylphosphinoethyltriethoxysilane to produce a diphenylphosphinoethyl-funtionalized silica.

Adsorption is preferably carried out by passing the impure acetic acid through a bed of phosphine-functionalized support. The invention may be carried out in a continuous or batch-wise fashion in accordance with known procedures. Continuous operation is preferred, as is the use of a plurality of adsorbent contact zones. When a plurality of adsorbent contact zones are used, one zone may be in use while adsorbent in a second zone is regenerated. The use of three contact zones is particularly preferred, with two zones in use at the same time, one a lead contact zone and the second a polishing zone, while the third zone is regenerated.

The adsorptive contact is conveniently carried out at moderate temperatures. Suitable temperatures are in the range of about 10° C. to 100° C., preferably 15° C. to 80° C. Flow rates of about 0.005 to 50 volumes of acetic acid per volume of adsorbent per hour are preferred, more preferably about 0.02-5. In general, slower feed flow rate reduces product impurity at a given bed-volume. Therefore, flow rate may be optimized depending on the volume of adsorbent utilized in the method.

The phosphine-functionalized support retains the iodide compound impurities adsorbed thereon and purified acetic acid can be separated. Initially, there can be substantially complete removal of the iodide compound impurities and the recovered acetic acid is of exceptional purity. Over the course of time the phosphine-functionalized support gradually becomes less effective for the removal of these impurities.

Thus, when the separation efficiency of the phosphine-functionalized support has fallen below a desired point, the adsorbent is preferably regenerated. The adsorbent is preferably regenerated by a high temperature thermal heating, followed by refunctionalization of the support with the phosphine. For instance, used 2-diphenylphosphinoethyl-functionalized silica may be oxidized and/or reduced at temperatures ranging from 250° C. to 500° C., and then refunctionalized with diphenylphosphinoethyltriethoxysilane to produce regenerated 2-diphenylphosphinoethyl-functionalized silica. It is advantageous to employ a plurality of parallel contact zones such that while one zone is being regenerated the feed is passed through a zone containing fresh or regenerated contact material so that optimum impurities removal can be achieved.

Following the purification method, a purified acetic acid product having decreased iodide compound impurities content of 10 ppb, or less, is recovered.

The following examples merely illustrate the invention. Those skilled in the art will recognize many variations that are within the spirit of the invention and scope of the claims.

### EXAMPLES

### Adsorption Runs

### Example 1

A solution (1.06 g) composed of 97.14 wt % acetic acid and 2.86 wt % methyl iodide is added to a 5 mL vial at 20° C. 2-Diphenylphosphinoethyl functionalized silica gel (0.30 g of 0.62 mmol/g functionalized gel) is then added to the vial. After an initial brief shaking, the vial is set aside and sampled by syringe several times over a period of 70 minutes. Sample analysis is carried out using a Nicolet 6700 FTIR equipped with a single bounce ATR accessory containing a zinc selenide crystal. Methyl iodide concentration in the 70 minute sample is 0.26 wt %.

### Example 2

The method of example 1 is repeated using a solution (1.08 g) composed of 97.12 wt % acetic acid and 2.88 wt % butyl iodide and 0.29 g of the 2-diphenylphosphinoethyl functionalized silica gel. Butyl iodide concentration in the 70 minute sample is 2.16 wt %.

### Example 3

The method of example 1 is repeated using a solution (2.58 g) composed of 96.1 wt % acetic acid and 3.9 wt % hexyl iodide and 0.51 g of a 1.05 mm/g 2-diphenylphosphinoethyl functionalized silica gel. The vial is set aside for 40 minutes and an aliquot of solution obtained after 40 minutes is analyzed using an

Analect Diamond 20 FTIR equipped with a 0.074 mm path length transmission cell which contained zinc selenide windows. Hexyl iodide concentration in the 40 minute sample is 3.4 wt %.

### Example 4

The method of example 3 is repeated using a solution (2.67 g) composed of 91.9 wt % acetic acid and 8.1 wt % iodobenzene and 1.04 g of the 2-diphenylphosphinoethyl functionalized silica gel. The vial is set aside for 35 minutes. lodobenzene concentration in the 35 minute sample was 7.0 wt %.

The results show that the phosphine-functionalized support effectively removes low levels of iodide from the acetic acid feed. Based on the results, approximate adsorption capacity for methyl iodide is about 90 mg/g, for butyl iodide is about 27 mg/g, for hexyl iodide is about 25 mg/g of support, and for iodobenzene is about 28 mg/g of support.

We claim:

1. A method of purifying acetic acid containing 15 to 100 ppb iodide compounds which comprises contacting the acetic acid in the liquid phase with a phosphine-functionalized support, and recovering a purified acetic acid product having 10 ppb, or less, iodide compounds.

2. The method of claim 1 wherein the phosphine-functionalized support is a tertiary phosphine-functionalized support.

3. The method of claim 2 wherein the tertiary phosphine is an alkyldiarylphosphine.

4. The method of claim 1. wherein the support is an inorganic oxide.

5. The method of claim 4 wherein the inorganic oxide is selected from the group consisting of silica, alumina, titania, zirconia, titania-silica, zirconia-silica, alumina-silica, and mixtures thereof.

6. The method of claim 1 wherein the acetic acid is produced by the carbonylation of methanol.

7. The method of claim 1 wherein the acetic acid contains 15-60 ppb iodide compounds.

8. The method of claim 1 wherein the iodide compounds are selected from the group consisting of methyl iodide, butyl iodide, hexyl iodide, iodobenzene, and mixtures thereof.

9. A method which comprises reacting methanol and carbon monoxide in the presence of a catalyst and methyl iodide to produce a crude acetic acid effluent, distilling the crude acetic acid effluent in one or more distillation columns to produce an acetic acid stream containing 15 to 100 ppb iodide compounds, contacting the acetic acid stream in the liquid phase with a phosphine-functionalized support, and recovering a purified acetic acid product having 10 ppb, or less, iodide compounds.

10. The method of claim 9 wherein the phosphine-functionalized support is a tertiary phosphine-functionalized inorganic oxide.

11. The method of claim 10 wherein the inorganic oxide is selected from the group consisting of silica, alumina, titania, zirconia, titania-silica, zirconia-silica, alumina-silica, and mixtures thereof.

12. The method of claim 10 wherein the tertiary phosphine is an alkyldiarylphosphine.

13. The method of claim 9 wherein the acetic acid stream contains 15-60 ppb iodide compounds.

14. The method of claim 9 wherein the iodide compounds are selected from the group consisting of methyl iodide, butyl iodide, hexyl iodide, iodobenzene, and mixtures thereof.
